(19) United States
(12) Patent Application Publication
(10) Pub. No.: US 2004/0229382 A1
(43) Pub. Date: Nov. 18, 2004

(54) Characterized arrangements of mixtures (CAM) using polymers

(76) Inventors: Ronald Breaker, Guilford, CT (US); Yung-Chi Cheng, Woodbridge, CT (US)

Correspondence Address: MORGAN LEWIS & BOCKIUS LLP, 1111 PENNSYLVANIA AVENUE NW, WASHINGTON, DC, 20004, US

(21) Appl. No.: 10/166,352

(22) Filed: Oct. 19, 2001

Related U.S. Application Data
(60) Provisional application No. 60/242,147, filed on Oct. 23, 2000.

(51) Int. Cl. G01N 33/545

(52) U.S. Cl. 436/531

(57) ABSTRACT

This invention relates to the characterization of mixtures. More specifically, this invention provides tools and methodologies for characterizing a mixture by detecting defined and undefined structures which form when the substances of the mixture contact a configuration of immobilized polymers.

### FIELD OF THE INVENTION

This invention relates to the characterization of mixtures. More specifically, this invention provides tools and methodologies for characterizing a mixture by detecting defined and undefined structures which form when the substances of the mixture contact a configuration of immobilized polymers.

### BACKGROUND OF THE INVENTION

All publications and patent applications herein are incorporated by reference to the same extent as if each individual publication or patent application was specifically and individually indicated to be incorporated by reference.

There is great interest in determining what substances are in mixtures. This is especially true with respect to the potency and quality control aspects related to foods, food supplements, herbal compositions, medical foods and drugs. Information on what substances are in various biological mixtures, such as cell and tissue extracts, is of major importance to medical diagnoses, medical treatments and forensic science. Also, information regarding the substances in the environment around us is of increasing importance to our safety and well being. In addition, information about the presence or absence of particular substances in industrial mixtures is of interest to both the manufacturer and the consumer. For example, it may be important to know whether certain mixtures contain substances such as, but not limited to, transgenes introduced into genetically modified organisms (GMOs), pesticides, herbicides, various natural and synthetic poisons, viruses, bacteria, growth hormones and certain beneficial or toxic herbal components.

Developing methods to identify the substances in a mixture presents unique technical problems, including low concentrations of active molecular components, unknown interfering constituents which can degrade the resolution of many screening systems currently available and the lack of specific probes to identify some substances.

### OVERVIEW OF THE INVENTION

As described below, the array or microchip technologies currently used for identifying substances in a mixture require specific, predetermined binding or hybridization between sites on tethered oligonucleotides and the molecules in the sample being analyzed or tested. The present invention provides novel methods which can be used for describing both defined and undefined structures generated when a mixture is exposed to a configuration of polymers, including an array of oligonucleotides. Thus, the methods of the present invention provide a more complete and unique characterization of a mixture than is previously available.

### SUMMARY OF THE INVENTION

The present invention provides novel methods of characterizing a mixture. More specifically, the present invention provides methods of characterizing a mixture which involves contacting a mixture with a configuration of immobilized polymers; detecting defined and undefined structures which result from the contact of the polymers and the substances of the mixture; and then describing the arrangement of the detected structures. The described arrangement of the detected structures provides a characterized arrangement of that particular mixture.

The present invention also provides methods of further characterizing a mixture by repeating the contacting, detecting and describing steps one or more additional times with the same mixture using different configurations of polymers. Thus, the present invention provides a set of resulting arrangements which characterize a particular mixture.

The present invention also provides methods of characterizing a mixture by repeating the contacting, detecting and describing steps one or more times with the same or a different mixture and then comparing the resulting arrangements.

The present invention further provides methods of characterizing mixtures which utilize a variety of different polymers, including, but not limited to, nucleic acids, proteins, antibodies, viruses, plastics, aptamers and tripartate multifunctional integrated constructs.

The present invention also provides methods of characterizing a mixture which utilize a configuration of immobilized polymers as an array, such as a microarray.

The present invention further provides methods of characterizing a mixture which utilize a configuration of immobilized polymers as an array on a silicon chip or in a crystalline colloidal array.

The present invention also provides methods of characterizing any kind of a mixture, including, but not limited to, botanical extracts, tissue homogenates, microbial extracts, plasma, urine, serum, blood, synthetic chemicals or optical isomers.

The present invention also provides methods of characterizing a mixture involving contacting a mixture with two or more configurations of immobilized polymers; detecting defined and undefined structures which result from the contact of the polymers and the substances of the mixture; and describing the arrangements of the detected structures, wherein the arrangements characterize the mixture.

The present invention also provides a system for characterizing a mixture which includes: 1) one or more configurations of immobilized polymers; 2) a mixture; 3) a means for exposing the mixture to the one or more configurations of polymers and detecting defined and undefined structures comprising the polymers and the substances comprising the mixture; and 4) a computer processor, including memory, for analyzing and storing the detection of the defined and undefined structures. The present invention further provides such a system which also includes a computer processor, including memory, for comparing the characterized arrangements with one or more previously-stored characterized arrangements for the same or a different mixture.

### BRIEF DESCRIPTION OF THE DRAWINGS

FIG. 1. The tripartite design for allosteric ribozyme construction.

A. Sequence and secondary structure for an FMN-sensitive allosteric ribozyme (Soukup and Breaker, Allosteric ribozymes. In Ribozymes: Biology and Biotechnology. Gaur and Krupp (eds.), Eaton Pub. (in press) (1999)). In this construct, the cm⁺FMN1 communication module (cm) is the first sequence class (1) that was previously identified to undergo allosteric activation (⁺) in the presence of flavin mononucleotide (FMN). Base-paired elements that are required for hammerhead ribozyme activity (I, II, and III) are labeled according to Hertel et al. (Nucleic Acids Res. 20:3252 (1992). An arrowhead identifies the site of hammerhead-mediated cleavage.

B. A tripartite construct carrying a randomized aptamer domain used as the pool to initiate in vitro selection. N₂₅ represents 25 nucleotides with random base identity.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

A. Definitions.

Arrangement. As used herein, an “arrangement” refers to the expression of a defined or undefined structure. Examples include, but are not limited to, graphs, tracings, 2D and 3D displays, patterns, fingerprints, bit maps, histograms, scatter plots and matrices.

Array. As used herein, “array” refers to an orderly set of two or more polymers.

Biopolymer. As used herein, a “biopolymer” refers to polymers found in biological systems. The biopolymers used for this invention may be naturally or artificially produced.

CAM. As used herein, “CAM” is an acronym for Characterized Arrangement of Mixtures.

CAM-Mediated Quality Assurance. As used herein, “CAM-Mediated Quality Assurance” refers to a quality assurance program or method which utilizes the CAM technology of the present invention.

CAM-Mediated Quality Control. As used herein, “CAM-Mediated Quality Control” refers to a quality control procedure or method which utilizes the CAM technology of the present invention.

Copolymer. As used herein, a “copolymer” is a polymer containing two different monomers. Examples of copolymers include, but are not limited to, dynel (acrylonitrile and vinyl chloride), SBR (styrene-butadiene rubber) and nucleoprotein (nucleic acid and protein).

Defined Structure. As used herein, a “defined structure” refers to a predictable or foreseeable binding of a polymer to a substance based on the known properties of that polymer. An example of a defined structure is where a known ligand binds to its cognate receptor leading to receptor activation.

Fluid. As used herein, a “fluid” refers to a liquid or a gas.

Monomer. As used herein, a “monomer” refers to a natural or synthetic molecule that can link with others to form a polymer. Examples include, but are not limited to, nucleotides, amino acids, acrylonitrile, vinyl chloride, isoprene, chloroprene, butadiene and isobutylene.

Macromolecule. As used herein, a “macromolecule” refers to a larger molecule consisting of many smaller units linked together.

Mixture. As used herein, a “mixture” refers to a composition of two or more substances that are not chemically combined with each other and are capable of being separated. Examples of mixtures include, but are not limited to, fats, carbohydrates, blood, urine, botanical extracts, herbal compositions, tissue homogenates, microbial extracts, plasma, synthetic chemicals and optical isomers.

Nucleic Acid. As used herein, a “nucleic acid” refers to a natural or synthetic polymer of two or more nucleotides (synonymous with nucleosides or mononucleotides).

Peptide. As used herein, a “peptide” refers to natural or synthetic polymer of two or more amino acids.

Polymer. As used herein, a “polymer” refers to a macromolecule made by linking together monomers. Examples of polymers include, but are not limited to, nucleic acids, oligonucleotides, DNA, RNA (mRNA, tRNA, ribosomes), proteins, peptides, oligopeptides, polypeptides, enzymes, abzymes, ribozymes, aptamers, starch, glycogen, viruses, viroids, antibodies, plastics, polyurethane, vinyl polymers (e.g., polyethylene), copolymers (e.g., dynel), latex, rubber, neoprene, polyisobutylene and copolymers.

Polymerization. As used herein, “polymerization” refers to the linking of two or more monomers to form a polymer.

Polymerize. As used herein, “polymerize” refers to undergoing polymerization.

Polymeric. As used herein, “polymeric” refers to something which has the properties of a polymer.

Substance. As used herein, a “substance” refers to anything that has mass and occupies space.

Undefined Structure. As used herein, an “undefined structure” refers to an unpredictable or unforeseeable interaction of a polymer with a substance. An example of an undefined structure is where a substance interacts with an enzyme outside of its active and allosteric site resulting in catalytic activity.

B. Characterizing Botanicals: One Example of How Current Methods Fail.

Botanicals, including herbs, have become a focal point for the identification of new active agents to treat diseases. Accordingly, active compounds, derived from plant extracts, are of continuing interest to the pharmaceutical industry. For example, taxol is an antineoplastic drug obtained from the bark of the western yew tree. It is estimated that approximately 50 percent of the thousands of drugs commonly used and prescribed today are either derived from a plant source or contain chemical imitations of a plant compound (Mindell, E. R., 1992, Earl Mindell's Herb Bible, A Fireside Book).

Currently, a number of medicinal formulations, food supplements, dietary supplements and the like contain herbal components or extracts from herbs. Herbal medicines have been used for treating various diseases of humans and animals in many different countries for a very long period of time (see, e.g., Kessler et al., 1996, The Doctor's Complete Guide to Healing Medicines, Berkley Health/Reference Books); Mindell, supra).

Screening methodologies for natural products can be broadly classified into two groups: bioassays and mechanism-based assays. Bioassays are the oldest, and so far most productive, screening tool. These assays measure the effect of natural samples on the viability or metabolism of disease-related cell types such as bacteria, fungi, viruses, and tumor cells. For example, the .beta.-lactam antibiotics (e.g., penicillins and cephalosporins) were discovered by testing microbial broths for bacterial growth inhibition in culture tests. Likewise, the antifingal compounds nystatin and amphotericin B were isolated from broths that inhibited growth of yeast in culture tests. However, mainly due to their lack of specificity and sensitivity, most bioassays have been replaced as primary screens with more sophisticated, mechanism-based assays.

Mechanism-based assays can be subdivided into three general categories: recombinant cell-based assays, enzymatic (biochemical) assays, and binding assays. These assays are designed with the need for high throughput capacity so they must be robust, simple, and amenable to automation in a parallel processing mode, including recombinant, cell-based assays screen for some known functional response.

Usually a target receptor, enzyme, or other protein is introduced into cultured cells by genetic engineering. Inhibition or induction of target activity is associated with an easily-measured response. For example, modifiers of transcription factors (TF) can be measured by fusing the TF's target DNA sequence (enhancer or promoter) to a luciferase (light-producing) gene. TF agonists result in transcription of the luciferase gene, and light is produced. If an antagonist is present, light is not produced. One advantage of cell-based assays over enzymatic and binding assays is that they may provide more physiologically appropriate leads because intact cells are used. On the other hand, these screens can be very difficult to develop, can be slow and are quite variable (Janzen et al., Society for Biomolecular Screening Meeting, Nov. 7-10, 1995).

Enzymatic assays are cell-free screens that directly or indirectly test the effect of soluble compounds on the activity of purified target molecules. For example, viral reverse transcriptase inhibitors can be screened by measuring the incorporation of radiolabeled thymidine into a growing DNA chain from a polyuridine RNA template. These assays can be very sensitive and are amenable to automation using microtiter plates. For natural product screening, however, unknown compounds in the samples can dramatically interfere with the results, leading to unacceptably high levels of false negatives and false positives. For example, greater than 15% of aqueous extracts from terrestrial plants, cyanobacteria, marine invertebrates, and algae exhibit positive activity in HIV antiviral screens due to interfering compounds such as plant tannins (Cardellina et al., J. Nat. Prod. 56:1123-1129, 1993).

Binding assays are particularly useful for screening soluble mixtures for compounds that bind, and thus potentially inhibit, target therapeutic molecules. The target molecule (usually a protein) can be affixed or tethered to a solid substrate such as the sides of microtiter wells, beads, or chromatographic supports. If the target molecule is a receptor, it can be expressed in the membrane of a cell, which is attached to the solid support. The samples are incubated with the immobilized targets, and bound ligands are detected, usually through an associated colorometric or fluorescent reaction. Alternatively, the sample is mixed with a soluble-phase target that is captured using an anti-target antibody. Such binding assays are advantageous as they facilitate the washing and isolation of target-ligand complexes. However, they suffer from several disadvantages, particularly for natural product screening. One problem is that multiple, weak-binding, background compounds, if present in sufficient quantities, can give a positive signal. Therefore, improved clean-up capability is desirable unless heavy washing is possible.

Another general problem with immobilized-target binding assays is that affixing target proteins to solid substrates often results in a functional change or inactivation of the protein. This problem could be addressed to some extent by inserting some inert “handle” such as a peptide epitope into the target by recombinant DNA technology. The protein-ligand complex could then be isolated through the use of an antibody to this epitope. However, developing these artificial targets is time-consuming and expensive. A disadvantage of the commonly used microtiter (ELISA) format is that the target molecule, which is usually attached to the well wall, is not in contact with most of the soluble sample dispersed throughout the well, which results in the need for increased reaction times. Some improvements have been made through using reduced reaction volumes.

The use of polymers (such as polypeptides, polynucleotides, oligonucleotides or modified oligonucleotides) that recognize small molecules and proteins has been described (see for example Norel et al., Comb Chem High Throughput Screen (1999) 2(4):223-37). However, these assays require that the constituents present in the natural substance recognize the polymer as a ligand or substrate; i.e., the binding is specific and selective. Assays in which both specific and selective functional properties are exploited are of limited use in the analysis of natural substances because the binding of the constituent to the polymer simply validates a previously recognized property. This presents an especially acute problem in the analysis of natural products, such as botanical extracts, since the functional characteristic properties of many of the constituents in most extracts is unknown. What is needed is an assay system which is not dependent on the foreknowledge of the functional properties of the constituent molecules; e.g., a binding assay where the binding is selective, yet non-specific. Such an assay may be accomplished by analyzing how constituent molecules of natural products bind to sets of polymers as a group, wherein the resulting binding pattern represents the distinctive characteristic or identifying property of the product.

Recent advances in oligonucleotide microarray technology make possible massive parallel screening of polymers. These arrays have been used to study cell-cycles, biochemical pathways, genome-wide expression in yeast, cell growth, cellular differentiation, cellular responses to a single chemical compound, and genetic diseases, including the onset and progression of the diseases (M. Schena et al., 1998, TIBTECH 16:301). However, no researchers to date, have attempted to apply these new technologies to screen for therapeutic natural products outside of a gene expression context.

In contrast to the methods described above, the present invention discloses methods for characterizing the arrangement of a mixture by detecting defined and undefined structures after a mixture is contacted with a configuration of immobilized polymers. In this manner, it will be possible to ascertain the substances in a mixture such as that of an herbal composition. As explained below, the methods of the present invention have utility for any type of mixture whether natural or synthetic or even where it is a combination of natural and synthetic substances.

C. An Overview of Currently-Used Arrays, Microarrays and BioChips.

An “array” or “microarray” generally refers to a grid system which has each position or probe cell occupied by a defined nucleic acid fragments also known as oligonucleotides. The arrays themselves are sometimes referred to as “chips”, “biochips”, “DNA chips” or “gene chips”. High-density DNA microarrays often have thousands of probe cells in a variety of grid styles.

A typical molecular detection chip includes a substrate on which an array of recognition sites, binding sites, or hybridization sites are arranged. Each site has a respective molecular receptor which binds or hybridizes with a molecule having a predetermined structure. The solid support substrates which can be used to form surface of the array or chip include organic and inorganic substrates, such as glass, polystyrenes, polyimides, silicon dioxide and silicon nitride. For direct attachment of probes to the electrodes, the electrode surface must be fabricated with materials capable of forming conjugates with the probes (Whitesides et al., Langmuir 6:87-96 (1990); Hickman et al., J. Am. Chem. Soc. 113:1128-1132 (1991)).

Once the array is fabricated, a sample solution is applied to the molecular detection chip and molecules in the sample bind or hybridize at one or more sites. The sites at which binding occurs are detected, and one or more molecular structures within the sample are subsequently deduced. Autoradiography of radiolabeled batches is a traditional detection strategy, but other options are available, including electronic signal transduction. DNA may be labeled to serve as a probe by altering a nucleotide which then serves as a replacement analogue in the nick translational resynthesis of double stranded DNA. The chemically altered nucleotides may then provide reactive sites for the attachment of immunological or other labels such as biotin (Gilliam et al., Anal. Biochem. 157:199(1986)). Another example uses ruthenium derivatives which intercalate into DNA to produce photoluminescence under defined conditions (Friedman et al., J. Am. Chem. Soc. 112:4960 (1990)).

Of particular interest are DNA chips for sequencing and diagnostic applications. A DNA chip includes an array of binding sites each having single-stranded DNA probes or like synthetic probes for recognizing a respective DNA sequence. A sample of single-stranded DNA fragments, referred to as target DNA, is applied to the DNA chip. The DNA fragments attach to one or more of the probes.

In sequencing applications, a sequence of nucleotide bases within the DNA fragment can be determined by detecting which probes have DNA fragments bound thereto. In diagnostic applications, genomic samples from an individual are screened with respect to a predetermined set of probes to determine if the individual has a genetically-inherited disease or a genetic predisposition to a disease. A gene probe assay is considered ideal if it is sensitive, specific and easily automatable (for a review, see Nickerson, Current Opinion in Biotechnology 4:48-51 (1993)).

For discussions, construction, methodologies and applications of oligonucleotide arrays, microarrays, DNA chips or biochips, see, for example, U.S. Pat. Nos. 5,445,934; 5,605,662; 5,631,134; 5,632,957; 5,653,939; 5,736,257; 5,741,644; 5,744,305; 5,795,714; 5,843,767; 5,847,019; 5,945,283; 5,945,286; 5,952,172; 5,968,728; WO 90/15070; Schena et al., Parallel human genome analysis: Microarray-based expression monitoring of 1000 genes, Proc. Natl. Acad. Sci. USA 93, 10614-10619 (1996); DeRisi et al., Exploring the Metabolic and Genetic Control of Gene Expression on a Genomic Scale, Science 278, 680-686 (1997); Wodicka, et al., Genome-wide Expression Monitoring in Saccharomyces cerevisiae, Nature Biotechnology 15, 1359-1367 (1997); DeRisi et al., Use of a cDNA Microarray to Analyze Gene Expression Patterns in Human Cancer, Nature Genetics 14, 457-460 (1996); Marshall et al., DNA Chips: An Array of Possibilities, Nature Biotechnology 16, 27-31(1998); Schena et al., Microarrays: Biotechnology's Discovery Platform for Functional Genomics, Tibtech 16, 301-306 (1998); Ramsay, DNA Chips: State-of-the-art, Nature Biotechnology 16, 40-44 (1998); Chee et al., Accessing Genetic Information with High-Density DNA Arrays, Science 274, 610-614 (1996); and Chen et al., Profiling Expression Patterns and Isolating Differentially Expressed Genes by cDNA Microarray System with Colorimetry Detection, Genomics 50, 1-12 (1998).

As described above, current array or microchip technologies are based on specific, predetermined bindings or hybridizations between the sites on tethered oligonucleotides and molecules in a sample. Thus, these prior art methods are limited to recognizing specific, predetermined structures. However, non-binding and non-hybridizing structures are also of interest in characterizing a mixture. The present invention provides methods which characterize both defined and undefined structures which are described when a mixture is exposed to an array of polymers, including an oligonucleotide array.

Following are detailed descriptions of representative types of polymers which are utilized in the present invention.

D. Nucleic Acid Polymers.

Polymer arrays of nucleic acid probes can be used to extract information from, for example, nucleic acid samples. These samples are exposed to the probes under conditions that permit binding. The arrays are then scanned to determine to which probes the sample molecules have interacted with the nucleic acids of the polymer array. One can obtain information by careful probe selection and using algorithms to compare patterns of interactions. For example, the method is useful in diagnostic screening for genetic diseases or for the presence or identity of a particular pathogen or a strain of pathogen. For example, oligonucleotide arrays can be used to examine a nucleic acid sample from the virus to determine what strain it belongs to.

In typical applications, a complex solution containing one or more substances to be characterized contacts a polymer array comprising nucleic acids. For example, the array is comprised of nucleic acid probes. The probes of the array can be either DNA or RNA, which may be either single-stranded or double-stranded. In a preferred embodiment of the invention, the probes are arranged (either by immobilization, typically by covalent attachment, of a pre-synthesized probe or by synthesis of the probe on the substrate) on the substrate or chips in lanes stretching across the chip and separated, and these lanes are in turned arranged in blocks of preferably five lanes, although blocks of other sizes will have useful application. The present invention provides individual probes, sets of probes, and arrays of probe sets on chips, in specific patterns which are used to characterize the substances in a complex mixture by producing a distinct image which is representative of the binding interactions between the probes on the chip and the substances in the complex mixture. The pattern of hybridization to the chip allows inferences to be drawn about the substances present in the complex mixture.

The substances in the complex solution will bind to the nucleic acids on the array. The substances of the complex mixture which bind to the nucleic acids of the array may include, but are not limited to, complementary nucleic acids, non-complementary nucleic acids, proteins, antibodies, oligosaccharides, etc. The types of binding may include, but are not limited to, specific and non-specific, competitive and non-competitive, allosteric, cooperative, non-cooperative, complementary and non-complementary, etc. For example, the nucleic acids of the array can bind to complementary nucleic acids in the complex mixture but can also bind in a tertiary manner, independent of base pairing, to non-complementary nucleic acids.

The nucleic acids of the array or the substances of the complex mixture may be tagged with a detectable label. The detectable label can be, for example, a luminescent label, a light scattering label or a radioactive label. Accordingly, locations at which substances interact can be identified by either determining if the signal of the label has been quenched by binding or identifying locations where the signal of the label is present in cases where the substances of the complex mixture have been labeled. Based on the locations where binding is detected, information regarding the complex mixture can be obtained.

The methods of this invention will find particular use wherever high through-put of samples is required. In particular, this invention is useful in clinical settings and for determining the composition of complex mixtures. For example, in a pathological state such as cancer, the characteristic arrangement of nucleic acids bound to components in a patient's serum may be used for diagnosis and prognosis.

E. Protein Polymers.

Polypeptides are an exemplary system for exploring the relationship between structure and function in biology. When the twenty naturally occurring amino acids are condensed into a polymeric molecule they form a wide variety of three-dimensional configurations, each resulting from a particular amino acid sequence and solvent condition. For example, the number of possible polypeptide configurations using the twenty naturally occurring amino acids for a polymer 5 amino acids long is over three million. Typical proteins are more than 100 amino acids in length.

In typical applications, a complex solution containing one or more substances to be characterized contacts a polymer array comprising polypeptides. The polypeptides of the invention can be prepared by classical methods known in the art, for example, by using standard solid phase techniques. The standard methods include exclusive solid phase synthesis, partial solid phase synthesis methods, fragment condensation, classical solution synthesis and recombinant DNA technology. See, e.g., Merrifield, 1963 (Am. Chem. Soc. 85:2149-2152). On solid phase, the synthesis is typically commenced from the C-terminal end of the peptide using an alpha-amino protected resin. A suitable starting material can be prepared, for instance, by attaching the required alpha-amino acid to a chloromethylated resin, a hydroxy-methyl resin or a benzhydrylamine resin.

The alpha-amino protecting groups are those known to be useful in the art of stepwise synthesis of peptides. Included are acyl type protecting groups, aromatic urethane type protecting groups, aliphatic urethane protecting groups and alkyl type protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride, dimethyl formamide (DMF) mixtures.

These procedures can also be used to synthesize peptides in which amino acids other than the twenty naturally occurring, genetically encoded amino acids are substituted at one, two, or more positions of any of the compounds of the invention. For instance, naphthylalanine can be substituted for tryptophan, facilitating synthesis. Other synthetic amino acids that can be substituted into the peptides of the present invention include L-hydroxypropyl, L-3,4-dihydroxyphenylalanyl, d-amino acids such as L-d-hydroxylysyl and D-d-methylalanyl, L-α-methylalanyl and β-amino acids non-naturally occurring synthetic amino acids can also be incorporated into the peptides of the present invention (see Roberts et al., 1983, Peptide Synthesis 5:341-449).

One can replace the naturally occurring side chains of the twenty genetically encoded amino acids (or D amino acids) with other side chains, for instance with groups such as alkyl, lower alkyl, cyclic four, five, six, to seven-membered alkyl, amide, amide lower alkyl, amide di(lower alkyl), lower alkoxy, hydroxy, carboxy and the lower ester derivatives thereof, and with four, five, six, to seven-membered heterocyclic. In particular, proline analogs in which the ring size of the proline residue is changed from five members to four, six or seven members can be employed. Cyclic groups can be saturated or unsaturated, and if unsaturated, can be aromatic or non-aromatic. Heterocyclic groups preferably contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl, oxazolyl, piperazinyl, piperidyl, pyranyl, pyrazinyl, pyrazolidinyl, pyrazolinyl, pyrazolyl, pyridazinyl, pyridyl, pyrimidinyl, pyrrolidinyl, pyrrolinyl, pyrrolyl, thiadiazolyl, thiazolyl, thienyl, thiomorpholinyl and triazolyl. These heterocyclic groups can be substituted or unsubstituted. Where a group is substituted, the substituent can be alkyl, alkoxy, halogen, oxygen, or substituted or unsubstituted phenyl.

One can also readily modify the peptides of the instant invention by phosphorylation (see Bannwarth et al., 1996 Biorg. Med. Chem. Let. 6:2141-2146) and other methods for making peptide derivatives of the compounds of the present invention are described in Hruby et al., 1990 (Biochem. J. 268:249-262). Thus, the peptide compounds of the invention also serve as a basis to prepare peptide mimetics with similar biological activity. The array can also comprise peptide mimetics with the same or similar desired biological activity as the corresponding peptide compound but with more favorable activity than the peptide with respect to solubility, stability, and susceptibility to hydrolysis and proteolysis (see Morgan et al., 1989, Ann. Rep. Med. Chem. 24:243-252).

Peptides suitable for use in this embodiment generally include those peptides, for example, ligands, that bind to a receptor, such as the interleukin receptors, growth factor receptors, cytokine receptors, G-protein-linked receptors, the cell surface receptors, etc. Such peptides typically comprise about 150 amino acid residues or less and, more preferably, about 100 amino acid residues or less.

The peptides of the present invention may exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues may also be substituted with a homocysteine. Other embodiments of this invention provide for analogs of these disulfide derivatives in which one of the sulfurs has been replaced by a CH₂ group or other isostere for sulfur. These analogs can be made via an intramolecular or intermolecular displacement, using methods known in the art.

F. Antibody Polymers.

Antibodies, because of their inherent specificity, are natural candidates for use in polymers arrays present on the chip. Chips utilizing antibodies are sometimes classified as immunosensors. The binding of antigen to immobilized antibody is considered to be essentially irreversible within the first few hours (Stenberg et al., 1988, J. Immunol. Methods, 113:3-7). The characterization of complex mixtures relies on the interaction of substances in the mixture to the antibodies of the array. Detection of these interactions is an indication of the presence of both defined and undefined chemical or biological species (antigen). The concept of the invention lies in being able to monitor both defined and undefined binding interactions instead of just detecting defined interactions.

An extensive range of antibodies applicable for use in this invention are commercially available or can be made from descriptions of methods of preparation available in the literature. Linscott's Directory provides the most complete single listing of commercially available antibodies. Any antibodies described in the literature can be employed or adapted to the antibody array for characterization of a wide range of samples.

The method can be used to detect specific components of body fluids such as lipoproteins, cholesterol, albumin, alpha-1 antitrypsin, beta-2 microglobulin, ceruloplasmin, C1 inhibitor, C1 q and other complement components, C-reactive protein, cryofibrinogen, ferritin, meylin basic protein, transferrin, insulin, human chorionic gonadotropin, estrogen, progesterone or antigens shed from pathogens such as, H. influenza, N. meningitides, S. pneumonia, Bordefella pertussis, Borrelia burgdorferi (Lyme's disease), staphlococcus, and streptococcus.

G. Recombinant Protein Polymers Used as Viral Receptors.

Polymer arrays of recombinant protein probes can be used to derive information from viruses found in human serum samples. These samples are exposed to the arrays of recombinant proteins which are known receptors for specific viruses under conditions that permit binding. The arrays are then scanned to determine to which viruses in the sample have interacted with the recombinant protein of the polymer array. One can obtain information by careful probe selection and using algorithms to compare patterns of interactions. The method is useful in diagnostic screening for genetic diseases or for the presence or identity of a particular pathogen or a strain of pathogen. For example, a polymer array comprising recombinant CXCR-4, a cofactor receptor for certain strains of HIV-1, can be used to detect viruses which are capable of interacting with this receptor. Detection of such strains can be used to diagnose onset and progression of AIDS in HIV-1 infected individuals because the presence of CXCR-4 utilizing strains in an individual's serum is indicative of AIDS progression.

The recombinant viral receptor is preferably immobilized on a recombinant protein array on the surface of a chip, especially via an anti-receptor antibody which is used as a captor. Dilutions of human sera are placed in contact with the immobilized viral receptor array on the surface of the chip and the interaction of viral coat proteins with viral receptor proteins on the array is detected using any means standard in the art.

H. Plastic Polymers.

In a preferred embodiment, polymers can be derived from plastic materials such as rubber, latex, including acrylic and vinylic monomers. For example, Molecularly Imprinted Polymers (MIPs) can be generated. These MIPs mimic specific recognition in a manner similar to antibodies (Haupt et al., TIBTECH (1998) 16(11):468-475). Such “plastic antibodies” are generated by the formation of recognition sites by allowing polymerization of monomers around a target template or imprint molecule. The monomers carry selected functional groups that arrange around the template through either covalent or non-covalent interactions. With a high degree of crosslinking the monomers are then frozen into position by polymerization and the templates are removed by cleavage or solubilization. In a preferred embodiment, the MIPs are immobilized such that they can interact with substances comprised in various mixtures such botanicals and other biological fluids such as blood and urine.

Polymers derived from plastics can be used which have many properties that allow for detection of bound moieties. For example, photosensitive polyurethanes for use in ion-selective field effect transistor transducers (Muñoz et al., Biosen Bioelec (1007) 12(7):577-585), pyroelectric sensors made from electrically poled polymer films such as poly(vinylidene) and copolymer poly(vinylidene fluoride-co-trifluor ethylene) containing pyroelectrically active groups have been established (Brehmer et al., EXS (1997) 80:155-165). These polymers can be used to produce surfaces such as thin films where a change in pyroelectric signal caused by enzymatic reactions can be used as a means of detecting substances in complex mixtures.

I. Crystalline Colloidal Arrays (CCA).

Other sensors include crystalline colloidal arrays (CCA) (Holtz et al., Nature (1997) 389(6653):829-832). In this system solute selective functional groups can be used to create switchable optical diffraction devices consisting of a CCA polymerized within a hydrogel that responds to selective binding of particular molecules within the gel network (Asher et al., U.S. Pat. No. 5,854,078). At specific concentrations, CCA volume changes such that wavelength increases are observed with increasing concentration of solutes. Such changes are visible to the naked eye. A variety of multiple-functional groups can be attached to the polymer chains that interact with specific solute species. Each functionally distinct CCA would then be assorted in a specific configuration where wavelength shifts would be detected across the assortment of CCAs to generate characteristic patterns. For example, a complex mixture is added to a configuration comprising CCAs having exclusive functional groups which recognize separate solutes. The pattern of diffraction obtained from the complex mixture is recorded. The configuration is then treated with a mixture of solutes for which the functional groups are selective. Any area of the configuration comprising the CCA which is resistant to diffraction changes in the presence of the functional group solutes will also be recorded. The pattern of diffraction/non-diffraction in the presence and absence of the functional group solutes would be used to characterize the complex mixture, as the non-diffraction pattern in the presence of the solutes would comprise detection of undefined structures.

J. Aptamer Polymers.

Aptamers are oligonucleotides selected, in vitro, to specifically bind corresponding molecules. For example, biosensors have been developed that utilize immobilized nucleic acid aptamers to specifically detect free nonlabled non-nucleic targets such as proteins.

In one embodiment of the present invention, a configuration of immobilized aptamers is contacted with a mixture and the defined and undefined structures formed between the polymers and the substances of the mixture are detected. The described arrangement of the detected structures characterizes the mixture. The detection can be accomplished by methods well known to those skilled in the art and described elsewhere herein. These methods include, but are not limited to, fluorescent labeling (Holeman et al., Fold Des. 3(6):423-431 (1998); electrochemiluminescence detection (Bruno and Kiel, Biosens. Bioelectron. 14(5):457-464 (1999); and physical signals such as radiation and temperature changes (WO 98/27104).

For descriptions of the construction of aptamers, aptamer arrays and their use as biosensors, see, for example, Tucker et al., J. Chromatogr. B. Biomed. Sci. Appl. 732(1):203-212 (1999); Wang et al., Biochemistry 35(38):12338-12346 (1996); Lato and Ellington, Mol. Drivers 2(1-2):103-110 (1996); Feigon et al., Chem. Biol. 3(8):611-617 (1996); Bridonneau et al., J. Chromatogr. B. Biomed. Sci. Appl. 726(1-2):237-247 (1999); Famulok and Mayer, Curr. Top. Microbiol. Immunol. 243:123-136 (1999); S. D. Jayasena, Clin. Chem. 45(9):1628-1650 (1999); Romig et al., J. Chromatogr. B. Biomed. Sci. Appl. 731(2):275-284 (1999); Tang and Breaker, Chem. Biol. 4(6):453-459 (1997); Potyrailo et al., Anal. Chem. 70(16):3419-3425 (1998); German et al., Anal. Chem. 70(21):4540-4545 (1998); and B. E. Eaton, Curr. Opin. Chem. Biol. 1(1):10-16 (1997).

K. Aptamer and Protein Copolymers.

Many aptamers are involved in the direct interaction of oligonucleotides with protein, wherein such interaction is responsible for the biological effect of pharmacologically active oligodeoxynucleotides. For example, it has been established that the interferon-gamma-inhibitory aptamer oligonucleotide causes significant changes in secondary and tertiary structures of interferon gamma (Balasubramanian et al., Mol Pharmacol (1998) 53(5):926-932). Similar demonstrations of adaptation by proteins of aptamer sequence fit within the tertiary folds of the proteins has been seen with HIV-1 Rev peptide class II RNA aptamers (Ye et al., Chem Biol (1999) 6(9):657-669). Thus, as aptamer have the ability to significantly alter the structure of the proteins they interact with, in vitro selection of aptamers to identify new aptamer domains can be used to generate new ligand/aptamer interactions to induce changes in known protein targets. The conformational changes using these systems are readily measured by circular dichromism, fluorescence spectroscopy, and antibody binding (Balasubramanian et al., 1998).

L. Polymers Containing Aptamers.

Aptamer-Containing Polymers. Natural ribozymes, artificial ribozymes and deoxyribozymes that have been isolated by in vitro selection are not known to operate as allosteric ribozymes. In a preferred embodiment small-molecule effectors bind to ribozyme and deoxyribozyme domains and modulate catalytic rate. For example, using rational design strategies, a hammerhead self-cleaving ribozyme is coupled to different aptamer domains to produce ribozymes having rates of catalysis that can be controlled by specific ligands (see WO 98/27104). It is possible to construct, using a mix of in vitro selection and rational design strategies, biosensors that rely on nucleic acid sensor elements. This is accomplished by appending RNA or DNA aptamers to ribozymes and deoxyribozymes, thereby creating new enzymes having catalytic rates that can be influenced by specific chemical effectors (e.g., molecules of diagnostic interest), physical signals, and combinations thereof.

In a preferred embodiment, purified functional DNA polynucleotides that exhibit allosteric properties that modify a function or configuration of the polynucleotide with a chemical effector, a physical signal, or combinations thereof, are constructed. In some embodiments, the DNA is an enzyme exhibiting allosteric properties that modify the rate of catalysis of the enzyme. The invention further provided purified functional RNA and DNA polynucleotides having catalytic properties with rates that can be controlled by a chemical factor, a physical signal, or combinations thereof. In some embodiments, the polynucleotides contain from about 10 to about 100 bases, others are much larger.

Tripartate Multifunctional Integrated Constructs Used as Polymers. Rational modular design has lead to the development of complex-function biopolymers, including those comprising RNA subdomains. For example artificial ribozymes have been designed that are modulated by the binding of specific small organic molecules such as nucleotides. These artificial molecules can be constructed from independent domains; e.g., a ribozyme domain and an “aptamer” or ligand specific domain. Such a construct exploits the adaptive binding (aptamer) domain property of changing conformation upon ligand binding which can trigger the adjoining catalytic domain.

Polynucleotide sensors are designed and constructed independently or together to comprise the actuator domain and receptor domain in communication with the bridging domain such that binding of a ligand to the receptor domain and/or a signal triggers a conformational change in the bridging domain which modulates the activity of the actuator domain.

In one embodiment, a sample is incubated with the polynucleotide or device comprising the polynucleotide as a sensing element for a time under conditions sufficient to observe the catalytic or reporter effect produced by the aptamer domain. This is monitored using any method known to those skilled in the art, such as measurement and/or observation of polynucleotide self-cleavage or ligation; binding of a radioactive, fluorescent, or chromographic tag; binding of a monoclonal or fusion phage antibody; or change in component concentration, spectrophotometric, or electrical properties. It is an advantage of the invention that current biosensor technology employing potentiometric electrodes, field effect transistors (FETs), various probes and redox mediators can be adapted for use in conjunction with the new polynucleotide sensors of the invention for measurement of changes in polynucleotide function or configuration initiated by the aptamer domain.

Sensors can be engineered to detect any type of ligand such as, but not limited to, all types of organic and inorganic compounds, metal ions, minerals, macromolecules, polymers, oils, microbial or cellular metabolites, blood or urine components, other bodily fluids obtained form biological samples, pesticides, herbicides, toxins, nonbiological materials, and combinations of any of these. Organic compounds include various biochemicals in addition to those mentioned above such as amino acids, peptides, polypeptides, nucleic acids, nucleosides, nucleotides, sugars, carbohydrates, polymers, and lipids. One or more ligands may be sensed by the same sensor in some embodiments.

The invention correspondingly provides methods for characterizing mixtures by contacting mixtures with polynucleotide sensors. Use of sensors responsive to more than one ligand and/or signal, tandem use of an array of multiple sensors each responsive to different ligands and/or signals, and tandem use of the multiple sensors with sensors responsive to more than one ligand and/or signal, in many cases attached to a solid support, are encompassed by the invention.

In a related aspect, a variety of different chromatographic resins and coupling methods can be employed to immobilize sensors of the invention on a support. For example, a simple non-covalent method that takes advantage of the strong binding affinity of streptavidin for biotin as previously described (WO 98/08974)) may be employed. In other embodiments, sensors can be coupled to the column support via covalent links to the matrix, thereby creating a longer lived biosensor. Various parameters of the system including temperature, sample preparation, sensor size and sensitivity, and the like, can be adjusted to give optimal sensing properties. In fact, these parameters can be preset based on the kinetic or other characteristic displayed by the immobilized sensor.

Upon stimulation by a ligand or signal, the aptamer domain modifies its catalytic function or reporter function. Any observation of a change in polynucleotide-configuration or function may be employed to determine this. In many embodiments, an observation of a chemical reaction is made such a measurement and/or observation of polynucleotide self-cleavage or ligation, substrate cleavage, or generation of a catalytic reaction product using standard assays. In others, simple binding of a radioactive, florescent, or chromophoric tag, binding of a monoclonal or fusion phage antibody or binding of a tagged antibody is observed. Alternatively, changes in component concentration, temperature, pH, appearance, spectrophotometric or electrical properties and the like, may be observed.

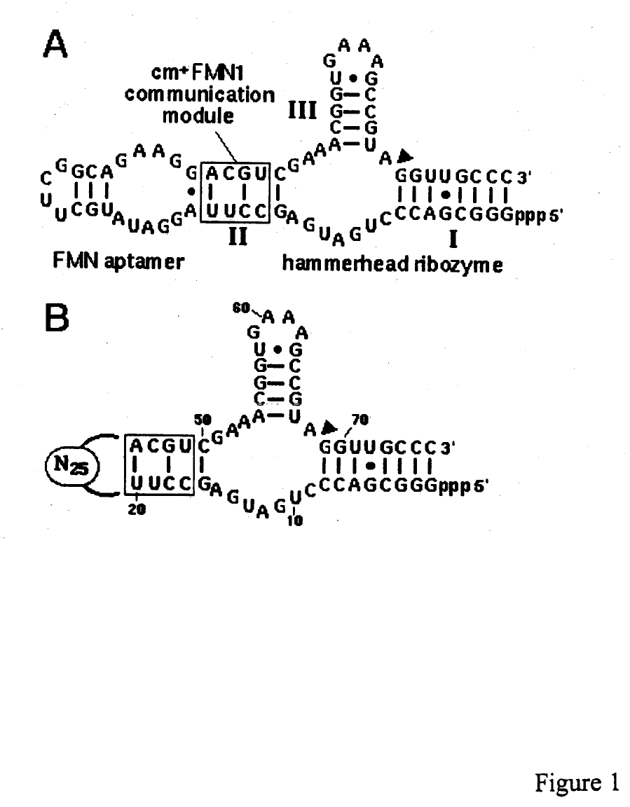

Sensors of the invention are developed through allosteric selection. Allosteric selection is an in vitro selection technique for the development of allosteric nucleic acid enzymes that are controlled by ligands for which an aptamer has not previously been identified. In this capacity, allosteric selection also represents a novel approach to the generation of aptamers that and target ligands. For this purpose, a random sequence library is appended to a catalytic nucleic acid motif such as the hammerhead ribozyme illustrated in FIG. 1. The random domain may be attached directly to the ribozyme (FIG. 1A) or through existing “communication modules” (FIG. 1B). In the latter case, the communication module is expected to inhibit self-cleavage within the ribozyme domain in the absence of a target ligand. In this manner, in vitro selection for self cleavage is the presence of target ligands will yield new aptamers and allosteric ribozymes if ligand binding to unique sequences derived form the random region triggers a conformational change that is conductive to ribozyme cleavage.

Multidomain polynucleotide molecule sensors of the invention may be RNA, RNA analogues, DNA, DNA analogues, or mixtures thereof. Analogues include chemically modified bases and unusual natural bases such as, but not limited to, 4-acetylcytidine, 5-(carboxyhydroxymethyl) uridine, 2′-O-methylcytidine, 5′-carboxymethylaminomethyl-2-thiouridine, 5-carboxymethylaminomethyluridine, dihydrouridine, 2′-O-methylpseudouridine, β-D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methylguanosine, 2-methyladenosine, 3-methyl cytidine, 5-methylcytidine, N6-methyladenosine, 7-methylguanosine, 5-methylaminomethyluridine, 5-methylaminomethyl-2-thiouridine, β-D-mannosylqueosine, 5-methylcarbonyhlethyluridine, 5-metyloxyuridine, 2-methyl-thio-N-6-isopentenyladenosine, N((9-β-Dribofuranosyl-2-methoythiopurine-6-yl)carbamoyl_threonine, N-((9-β-D-ribofuranosylpurine-6-yl)N-methyl-carbamoyl)threonine, uridine-5-oxyacetic acid methyl ester, uridine-5-oxyacetic acid, wybutoxosine, pseudouridine, queosine, 2-thiocytidine, 5-methyl-thiouridine, 2-thiouridine, 4-thiouridine, 5-methyluridine, N-((9-β-D-ribofuranosypurine-6-yl)carbamoyl)threonine, 2′-O-methyl-5-methyluridine, 2′-O-methyluridine, wybutosine, and 3-(3-amino-3-carboxypropyl)uridine.

M. Viral/Phage/Bacterial Polymers.

In another preferred embodiment, the configuration of immobilized polymers consists of viral, phage and/or bacterial display libraries.

In one example, foreign DNA fragments can be inserted into a minor coat protein gene of filamentous phage, creating a fusion protein that is incorporated into the virion. The fusion protein is incorporated into the virion, which retains infectivity and displays the foreign amino acids in an immunologically accessible form. The foreign amino acids are displayed on the surface, allowing fusion phage bearing antigenic determinants from a mixture to be identified. Defined and undefined structures resulting from contacting a mixture with such a phage display library can be identified using the techniques discussed above and in the examples below for Antibody Polymers and Nucleic Acid Polymers.

The generation and use of viruses, phages or bacteria which express chimeric binding proteins are well known to those skilled in the art. See, for example, Parmley and Smith, Gene 73:305-318 (1988); Daugherty et al., Protein Eng. 12(7):613-621 (1999); Gough et al., J. Virol. Methods 79(2):169-180 (1999); Gao et al., Proc. Natl. Acad. Sci. USA 96(11):6025-6030 (1999); Kay et al., Phage Display of Peptides and Proteins: A Laboratory Manual, Academic Pr. (1996); and U. Winkler, Bacterial Phage & Molecular Genetics, Springer Verlag (1976). Also, see, for example, U.S. Pat. Nos. 5,403,484, 5,571,698; 5,885,793; 5,824,520; 5,821,047; 5,702,892; 4,434,478; 4,348,477; and 5,403,484.

N. Polymers Used for Diagnostic and Prognostic Indications.

The clinical setting requires performing the same test on many patient samples. Automated methods of this invention lend themselves to these uses when the test is one appropriately performed on a biological chip. For example, a nucleic acid array can determine the particular strain of a pathogenic organism based on characteristic arrangement of nucleic acids bound to nucleic acids and proteins associated with the organism. The advanced techniques based on these assays now can be introduced into the clinic. Fluid samples from several patients are introduced into the test wells of a biological chip plate and the assays are performed concurrently.

In some embodiments, it may be desirable to perform multiple tests on multiple patient samples concurrently. According to such embodiments, rows (or columns) of the microtiter plate will contain probe arrays for diagnosis of a particular disease or trait. For example, one row might contain probe arrays designed for a particular cancer, while other rows contain probe arrays for another cancer. Patient samples are then introduced into respective columns (or rows) of the microtiter plate. For example, one column may be used to introduce samples from patient one, another column for patient two, etc. Accordingly, multiple diagnostic tests may be performed on multiple patients in parallel. In still further embodiments, multiple patient samples are introduced into a single well. In a particular well indicator the presence of a genetic disease or other characteristic, each patient sample is then individually processed to identify which patient exhibits that disease or trait. For relatively rarely occurring characteristics, further order-of-magnitude efficiency may be obtained according to this embodiment.

### EXAMPLES

### Example 1

### Characterized Arrangement of Human Tissue Using Oligonucleotides

A nucleic acid chip can be used for characterizing DNA isolated from the tissue of a particular patient. Methods for generating high-density arrays of oligonucleotide probes have been described previously, for example, VLSIPS™ technology has provided methods for making very large arrays of oligonucleotide probes in very small arrays (see U.S. Pat. No. 5,143,854). One such chip comprises a set of oligonucleotide probes of varying length in the range of ten to thirty bases with varying arrangement on 250 μm² sites on an array one cm² in size and may be prepared by any method standard in the art (Vasiliskov et al., 1999, Biotechniques 27:592-598; Niemeyer et al., 1994, Nucleic Acids Res. 22:5530-5539). The 3′-end of the oligonucleotide is covalently attached to the chip surface. The base composition of each of the oligonucleotides on the chip is variable to facilitate interactions with multiple substances of the complex mixture.

The DNA to be tested is isolated from any human tissue, amplified by PCR and cloned into any suitable vector (see Maniatis et al., 1992, Molecular Cloning: A Laboratory Manual, Cold Spring Harbor Laboratory Press). DNA from the clones is again amplified by PCR, transcribed in vitro and labeled with fluorescein-UTP using phage RNA polymerase (fluorophore-labeled nucleotides are available from a variety of commercial suppliers). The RNA transcripts are then fragmented and allowed to interact with the nucleic acids comprising the polymer array on the chip. The sample transcripts are hybridized to the chip in a solution composed of 6×SSPE, 0.1% Triton x-100 for thirty minutes at 15° C. Transcripts which do not bind to the chip are removed by washing with excess buffer.

Following removal of unbound transcripts to the oligonucleotides of the chip surface, the chip is scanned by any means standard in the art such as confocal scanning fluorescence microscopy. To quantitate the data, pixel counts are measured within each site. The fluorescence intensity for each feature is scaled to a mean determined from the total number of signals generated on the array. Tertiary, undefined, non-complementary transcript-oligonucleotide interactions result in lower fluorescence intensity than defined perfectly matched complementary interactions (Fodor et al., 1998, U.S. Pat. No. 5,837,832). After scanning, the chip is stripped and rehybridized; samples are hybridized to the same chip to determine reproducibility.

The intensity of each signal along with the signals on the chip is determined to produce a unique pattern on the chip. This pattern consists of intense signals (above the mean) which are indicative of defined, complementary interactions and weaker signals (below the mean), which are indicative of non-complementary, tertiary interactions. The differences in the observed patterns are then correlated with differences in the cloned genomic DNA.

### Example 2

### Production of a Random Polypeptide Configuration

Protein arrays may be generated by any means standard in the art, such as that reported by Rava et al., 1999 (U.S. Pat. No. 5,874,219) and Dower et al., 1999 (U.S. Pat. No. 5,869,451). A surface bearing silane groups terminating in 6-nitroveratryloxycarboxamide (NVOC-NH) residues is prepared as a substrate for synthesis of the peptides on the surface. Active esters of glycine and phenylalanine protected at the amino group with NVOC are prepared as reagents.

For a monomer set of size n, (n×1) cycles are required to synthesize all possible sequences of length 1. A cycle consists of two elements, first, irradiation through an appropriate mask to expose the amino groups at the sites where the next residue is to be added, with appropriate washes to remove the by-products of the deprotection, and second, addition of a single activated and protected (with the same photochemically-removable group) monomer, which will react only at the sites addressed in the first step, with appropriate washes to remove the excess reagent from the surface.

The above cycle is repeated for each member of the monomer set until each location on the surface has been extended by one residue in one embodiment. In other embodiments, several residues are sequentially added at one location before moving on to the next location. This step is optionally followed by addition of a protecting group to stabilize the array for later testing. A final deprotection of the entire surface (removal of photoprotective side chain groups) may be required.

A surface is provided with regions masked and unmasked. The surface is exposed to a reagent containing glycine, with the resulting structure being a monomer comprising glycine. Thereafter, further regions are masked, and the surface exposed to a reagent containing phenylalanine, with the resulting structure comprising a polymer of glycine coupled to phenylalanine. The process proceeds, consecutively masking and exposing the sections as shown until the multiple structure of distinct polymers are obtained. The terminal groups are capped by acetylation and all possible trimers of glycine-phenylalanine are obtained.

It will be appreciated by those of skill in the art that the above method could readily be used to simultaneously produce thousands or millions of oligomers on a substrate using the techniques disclosed herein. Consequently, the method results in the ability to practically test large numbers of, for example, dipeptides, tripeptides, tetrapeptides, pentapeptides, hexapeptides, heptapeptides, octapeptides, dodecapeptides or larger polypeptides.

In synthesizing the dimer of an aminopropyl group and a fluorescent group, a functionalized durapore membrane is used as a substrate. The durapore membrane is a polyvinylidine difluoride with aminopropyl groups. The aminopropyl groups were protected with the DDZ group by reaction of the carbonyl chloride with the amino groups, a reaction readily known to those of skill in the art. The surface bearing these groups was placed in a solution of THF and contacted with a mask bearing a checkerboard pattern of opaque and transparent regions. The mask is exposed to ultraviolet light having a wavelength down to at least about 280 nm for about five minutes, although a wide range of exposure times and temperatures may be appropriate in various embodiments of the invention.

The surface of the membrane is then washed with a fluorescent label which includes an active ester bound to a chelate of a lanthanide. These materials fluoresce in the red and the green visible region. After the reaction with the active ester in the fluorophore was complete, the locations in which the fluorophore is bound can be visualized by exposing them to ultraviolet light and observing the red and the green fluorescence using any method standard in the art.

### Example 3

### Characterized Arrangement of Serum Using Random Peptides

The polypeptide chip described above may be used for characterizing substances in human serum. The amino acid composition of each of the peptide on the chip is variable to facilitate interactions with multiple substances of the complex mixture. The serum to be tested is isolated from human blood using any standard technique in the art.

Following incubation of the sera and removal of unbound substances to the polypeptides of the array, the chip is scanned by any means standard in the art such as confocal scanning fluorescence microscopy. To quantitate the data, pixel counts are measured within each site. The fluorescence intensity for each feature is scaled to a mean determined from the total number of signals generated on the array. After scanning, the chip is stripped and rehybridized; samples are hybridized to the same chip to determine reproducibility.

The intensity of each signal along with the signals on the chip is determined to produce a unique pattern on the chip. This pattern consists of intense signals (above the mean) which are indicative of defined interactions and weaker signals (below the mean), which are indicative of allosteric, non-competitive interactions. The differences in the observed patterns are then correlated with differences in the serum samples from a single patient or among multiple patients.

### Example 4

### Production of a Configuration of Antibodies

Antibody arrays can be produced using any standard means in the art, for example, see Cornell et al., (U.S. Pat. No. 5,766,960). IgG antibodies are purified from ascites fluid by chromatography on Protein A to a single band on SDS-polyacrylamide gel electrophoresis. F(ab)₂ fragments are prepared from pure antibodies by pepsin digestion. Cation-exchange chromatography is used to purify active F(ab)₂ fragments as established by a single band of 100,000 molecular weight mark on SDS-polyacrylamide gel electrophoresis. F(ab)′ are obtained from F(ab)₂ by modification using the method of Martin et al., 1981 (Biochemistry 20:4229-4230). F(ab)₂ are reduced with dithiothreitol for two hours and dithiothreitol removed by ultrafiltration. F(ab)′ possesses comparable antigen binding activities to F(ab)₂ and produces a single band at the 50,000 and 25,000 molecular weight markets when SDS electrophoresis is carried out with non-reducing and reducing conditions, respectively.

F(ab)₂ are radiolabeled with ¹²⁵I by the chloramine T method. ¹²⁵ I-F(ab)₂ were incorporated into the unlabeled F(ab)₂. Pepsin digestion of antibody and subsequent reduction of the resultant F(ab)₂ to F(ab)′ fragments produces a single reactive thiol group at the carboxyl terminus of the F(ab)′ molecule. Coupling of this thiol group to the lipid molecule is achieved via the reaction with a terminal chlorine on polyethylene oxide attached to the polymerisable lipid molecule. The monolayer of derivatized lipid is formed by spreading lipid in decane solution on an air-water interface in a Langmuir-Blodgett trough. The nylon peg substrate is dipped through the interface so that the hydrocarbon chains of the lipid interact with the surface of the substrate. The surface of the trough is cleaned of lipid before the substrate was quickly withdrawn and transferred to the F(ab)′ solution. The lipid-coated substrate is immersed into an aqueous solution F(ab)′ and the reaction between the specific thiol on the F(ab)′ and the chlorine of the lipid polyethylene oxide linker group is carried out for at least three hours at room temperature under nitrogen. ¹²⁵ I-F(ab)′ is used as a marker of the reaction as it is carried out on the lipid coated substrate.

The F(ab)′ linked lipid coated substrate is then transferred to a palladium-coated glass slide substrate. A coating of at least 10¹¹ Fab molecules per cm² was achieved after incubation times longer than ten hours as calculated from radioactivity measurements of ¹²⁵I-Fab.

### Example 5

### Characterized Arrangement of Serum Using Antibodies

The antibody chip described above may be used for characterizing substances in human serum. The F(ab)₂ antigen specificity for each fragment on the chip is variable to facilitate interactions with multiple substances of the complex mixture. The serum to be tested is isolated from human blood using any standard technique in the art.

Following incubation of the sera and removal of unbound substances to the F(ab)₂ fragments of the array, the chip is scanned for radioactivity by any means standard in the art such as exposure to x-ray film. To quantitate the data, pixel counts are measured within each site. The intensity for each feature is scaled to a mean determined from the total number of signals generated on the array by measuring the density of any particular signal using any means standard in the art such as a densitometer. After scanning, the chip is stripped and rehybridized; samples are hybridized to the same chip to determine reproducibility.

The intensity of each signal along with the signals on the chip is determined to produce a unique pattern on the chip. This pattern consists of intense signals (above the mean) which are indicative of defined interactions and weaker signals (below the mean), which are non-specific, undefined interactions. The differences in the observed patterns are then correlated with differences in the serum samples from a single patient or among multiple patients.

### Example 6

### Characterized Arrangement of an Herbal Mixture Using Tripartate Sensors

Engineering RNA sensors. Ligand-specific molecular sensors composed of RNA were created by coupling pre-existing catalysis receptor domains via structural bridges (see Soukup et al., Proc Natl Acad Sci USA (1999) 96:3584-35-98, hereby incorporated by reference). Binding of ligand to the receptor triggers a conformational change within the bridge, and this structural reorganization dictates the activity of the adjoining ribozyme. The modular nature of these tripartite constructs makes possible the rapid construction of precision RNA molecular sensors that trigger only in the presence of their corresponding ligand.

Oligonucleotides. Synthetic DNA and the substrate RNAs are prepared by standard solid phase methods and are purified by denaturing (8 M urea) polyacrylamide gel electrophoresis (PAGE) as described previously (WO 98/08974). RNA substrate was 5′-³²P-labeled with T4 polynucleotide kinase and (γ-³²P)-ATP, and repurified by PAGE. Double-stranded DNA templates for in vitro transcription using T7 RNA polymerase were generated by primer extension on a DNA template complementary to the desired DNA. Extension reactions are conducted with reverse transcriptase (RT) as described (Tang et al. Chem Biol (1997) 4:453-459).

In vitro selection. Selection for allosteric activation is performed by pre-selection each population sensor for self-cleavage in the presence and absence of specific ligands (e.g., cNTPs, FMN, small organic molecules etc.) in 10 μl of reaction buffer (50 mm Tris-Cl (pH 7.5 at 23° C.) and 20 mm MgCl₂ for 20 hours at 23° C. Section of constructs is punctuated at 5 hour intervals by heating samples to 65° C. for 1 minute to denature and refold any mis-folded molecules. Uncleaved RNA is purified by denaturing PAGE as above and purified from the gel by standard methods in the art (e.g., Sambrook et al., 1989). The resulting RNAs are selected by incubation in the reaction buffer in the presence of specific ligands for specific periods of time. Positive products are amplified by RT-PCR and the resulting double stranded DNA is transcribed in vitro to generate successive populations of RNA. RNAs are also generated which are inhibited in the presence of specific ligands.

In vitro selection of Allosteric Ribozymes. Populations of variant RNA composed of separate specific ligand binding aptamers (e.g., see FMN and cNTPs; 26, 37, 38), hammerhead ribozyme domains (Forster et al., Cell (1987) 49:211-220 and Fedor et al Biochemistry (1992) 31:12042-12054) comprising random sequence bridges are generated recombinantly (Soukup et al). The bridge replaces a majority of the natural “stem II” portion of the hammerhead motif (Tuschl et al., Proc natl Acad Sci USA (1993) 90:6991-6994 and Long et al., Proc Natl Acad Sci USA (1994) 91:6977-6981). The randomized domain within the tripartate construct confers either positive or negative allosteric control upon the adjoining aptamer domain. For any specific ligand, two or more pools comprising identical RNAs are subjected to in vitro selection for allosteric induction or inhibition. To isolate bridges that direct the allosteric induction, a negative selection for self-cleavage in the absence of the specific ligand is applied to one pool. RNAs that remain uncleaved during this reaction are isolated and then subjected to a positive selection for self cleavage in the presence of the same specific ligand. This method favors the isolation of ribozymes that activate only in the presence of the specific ligand. In contrast, a second pool is both transcribed and preselected in the presence of the ligand. The surviving RNA precursors are then subjected to positive selection in the absence of ligand, which favors the isolation of bridges that direct ribozymes to undergo allosteric inhibition.

Multidomain polynucleotide molecular sensor printing. Multidomain polynucleotide molecular sensors are obtained as described above for in vitro selection. The sensors are separately amplified using appropriate primers prior to application on nylon membranes (Chen et al., Genomics (1998) 51:313-324). Approximately 10 ng of each amplified target is applied on a positively charged nylon membrane using a PC (personal computer) controlled arraying system. Roughly 85,000 spots can be placed on a piece of nylon membrane measuring 35 by 55 mm using a 24-pin arraying tool.

The standardized protocol for the herbal extract preparation is as follows: The ingredients of herbal raw materials with proper ratios were placed in a jacketed reactor and extracted with water at an elevated constant temperature with mixing. The solid was separated from the liquid with a 120-mesh screen. The resultant filtrate was collected and then concentrated by evaporating the water under reduced pressure. The concentrated liquor was spray dried at elevated temperature to yield granulated powder. This bulk substance is then formulated into the desired form.

The herbal extract, at various concentrations, is added to a chip configuration comprising multidomain polynucleotide sensors, such variation in concentration to determine the optimal concentration for analysis is mechanical and routine. Further, such analysis is carried out in massively parallel fashion with multiple chips comprising multiple aptamer bearing sensors. The chip is allowed to incubate with sample in a buffer comprising 50 mm Tris-HCl (pH 7.5 at 23° C.) and 20 mm MgCl₂ for 10-20 minutes at 23° C. A probing means is used to discriminate between self cleaved/ligated and unperturbed ribozyme domains of sensors. The probing means can be an antibody (Reines D. Anal Biochem (1991) 196(2): 367-372 and Kitagawa et al., J Immunol Methods (1993) 164(1):5-11), chemical probes (Tuschl, et al., Curr Opin Struct Biol (1995) 5(3):296-302), and nucleic acid probes.

Arrangement of signals is determined by imaging means (e.g., a flatbed scanner or digital camera). The results comprise defined and undefined structures between polymers and substances of the complex mixture as self-cleavage/ligation can occur whether or not the aptamer domain is occupied.

Specific ligands for the sensors are then added to the chip treated with first probe. The chip is allowed to incubate with the ligands in 10 μl of reaction buffer (50 mm Tris-HCl (pH 7.5 at 23° C.) and 20 mM MgCl₂ for 10-20 minutes at 23° C. Subsequently, a second probe which is distinguished from the first, is added to discriminate between self-cleaved/ligated and unperturbed ribozyme domains remaining on the chip.

The arrangement of signals is determined by imaging means (see above). Any sites which are resistant to allosteric cleavage in the presence of specific ligands would comprise undefined structures. The arrangements of the images taken together is then be used to describe the complex mixture.

### Example 7

### Characterized Arrangement of Blood Using Aptamers

Aptamers for interferon-gamma are isolated by allosteric selection as outlined above, using cNTPs as ligands. After pools of RNAs are identified, the aptamer subdomains are amplified and cloned. Interferon gamma peptide is immobilized on a solid phase by standard methods (Schreiber et al., J Immunol (1985)134(3):1609-1618) and the aptamers are added to the immobilized protein as described in Balasubramanian et al. (1998).

Blood samples are isolated from diabetic and aged-sex matched controls. After removing erythrocytes by standard Ficoll-Paque gradients, serum is diluted in series in 10 mM Tris-Cl, EDTA, pH 7.5. The fractionated, diluted samples are then incubated with the immobilized aptamer-interferon complexes in the absence of ligand. A probing means is used to discriminate conformational changes in interferon gamma (see Balasubramanian et al., 1998). Arrangement of signals is determined by imaging means (e.g., a flatbed scanner or digital camera). The results comprise defined and undefined structures between polymers and substances of the diluted sample as conformational changes can occur whether or not the aptamer domain is occupied.

Subsequently, a second probe which is distinguished from the first, is added to discriminate between unperturbed polymers remaining on the chip.

The arrangement of signals is determined by imaging means (see above). Any sites which are resistant to conformational change in the presence of specific ligands would comprise undefined structures. The arrangements of the images taken together is then be used to describe the diluted blood sample (diabetic versus control).

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of characterizing a mixture comprising the steps of:
a) contacting a mixture with a configuration of immobilized polymers;
b) detecting defined and undefined structures comprising the polymers of step (a) and substances comprising the mixture; and
c) describing the arrangement of said detected structures, wherein the arrangement characterizes the mixture.

2. The method of claim 1 further comprising repeating steps a) through c) one or more times with the same mixture using different configurations of polymers, wherein the resulting arrangements characterize the mixture.

3. The method of claim 1 further comprising repeating steps a) through c) one or more times with the same or a different mixture and then comparing the resulting arrangements.

4. The method of claim 1 wherein the polymers are one or more polymers selected from the group consisting of nucleic acids, proteins, antibodies, viruses, plastics, aptamers and tripartate multifunctional integrated constructs.

5. The method of claim 1, wherein the configuration of immobilized polymers is an array.

6. The method of claim 1 wherein the configuration of immobilized polymers is on a silicon chip.

7. The method of claim 1 wherein the configuration of immobilized polymers is a crystalline colloidal array.

8. The method of claim 1 wherein the mixture is selected from the group consisting of botanical extracts, tissue homogenates, microbial extracts, plasma, urine, serum, blood, synthetic chemicals and optical isomers.

9. A method of characterizing a mixture comprising the steps of:
a) contacting a mixture with two or more configurations of immobilized polymers;
b) detecting defined and undefined structures comprising the polymers of step (a) and substances comprising the mixture; and
c) describing the arrangements of said detected structures, wherein the arrangements characterize the mixture.

10. A system for characterizing mixtures comprising:
a. one or more configurations of immobilized polymers;
b. a mixture;
c. a means for exposing the mixture to the one or more configurations of polymers and detecting defined and undefined structures comprising the polymers and the substances comprising the mixture; and
d. a computer processor, including memory, for analyzing and storing the detection of the defined and undefined structures.

11. The method of claim 10 further comprising:
e. a computer processor, including memory, for comparing the characterized arrangement obtained according to claim 10 with one or more previously-stored characterized arrangements for the same or a different mixture.
